(19) United States
(12) Patent Application Publication
(10) Pub. No.: US 2020/0000205 A1
(43) Pub. Date: Jan. 2, 2020

(54) NONWOVEN SOLID MATERIAL SUITABLE FOR TOPICAL APPLICATIONS

(71) Applicant: L'OREAL, Paris (FR)

(72) Inventors: Hiroyuki OGATA, Kawasaki-shi, Kanagawa (JP); Gaurav AGARWAL, Kawasaki-shi, Kanagawa (JP)

(21) Appl. No.: 16/489,024

(22) PCT Filed: Feb. 28, 2017
(86) PCT No.: PCT/JP2017/009111

§ 371 Date: Aug. 27, 2019

(51) Int. Cl. A45D 44/00; A61K 8/02; D04H 1/541

(52) U.S. Cl. CPC A45D 44/002; D04H 1/541; A61K 8/0212

(57) ABSTRACT

The invention relates to a nonwoven solid material, preferably a cosmetic or dermatological nonwoven solid material, comprising a plurality of fibers, wherein said fibers comprise a body comprising at least one polymeric material and a body coating comprising at least one hydrophilic polymer, said nonwoven solid material further comprising at least one releasable cosmetic ingredient or composition. The invention relates to a face mask, a manufacturing process and applications of said nonwoven solid material or face mask.

### FIELD OF THE INVENTION

The present invention relates to nonwoven solid material comprising a plurality of fibers. The present invention relates also to a method and process for preparing said nonwoven solid material and a method and process caring and/or improving a keratin material by application of said nonwoven solid material to the surface of said keratin material.

The present invention also relates to a nonwoven solid material having the form of a sheet, in particular a sheet mask.

The present invention relates more particularly to topical applications, and especially to cosmetic applications.

### BACKGROUND OF THE INVENTION

Sheet materials such as nonwovens are defined as fiber structures prepared by layering and entangling any kind of fibers or mix of them, such as natural fibers, synthetic fibers, glass fibers. Each substrate made up by such process has specific mechanical and physical properties depending on fiber characterization of individual fibers.

Particularly for cosmetic uses of such substrates natural fibers such as cotton, man-made fibers such as Viscose or blend of these cellulosic fibers with synthetic fibers are most common because of good liquid absorption capacity of the cellulosic materials.

On the other hand disadvantage of such cellulosic materials or their blend is poor desorption capacity (or delivery capacity) of the liquid to the skin. Desorption capacity here means the capacity of substrate to transfer the liquid from surface to skin.

Another drawback of using such cellulosic material for facial mask application is that lot of formulation impregnated is dripped down in packaging and results in wastage of formulation.

Usually, sheet masks are packaged in a folded format, typically in a sachet.

Yet another drawback of application of cellulosic material is that it is difficult for users to unfold the wet sheet mask because individual layer of such materials adhere to each other in wet state.

Tissue mask is the most common examples of substrate based cosmetic products, where most of the liquid formulation is retained within the substrate structure after use of product. It results in wastage of product and such substrate does not provide it without achieving the maximum efficacy.

Especially in case the tissue mask is impregnated with high amount of formulation, the most common range of impregnation being 15 to 35 grams liquid/mask, it is important that maximum amount of formulation can be transferred to skin to achieve its maximum efficacy.

Therefore, there is a need that such a sheet material can deliver maximum amount of formulation to skin.

In addition, for sheet mask applications it is important that it is convenient for user to unfold the mask and able to apply on face. For easy handling purpose and to make sure that the sheet mask is not damaged during the different steps of use; there is a need to find a material which is easy to unfold.

Also the sheet masks are commonly used as disposable products. Nonwoven sheets are very common material because of these are cost effectives and easily available. In order to have sufficient durability during use, these materials have generally a density of 40 to 70 gsm materials.

For the development of substrates for the cosmetic industry, specifically for facial mask applications, one important aspect is the capacity of substrate to absorb the liquid and hold it. Nonwoven materials of cellulosic fibers and its blend made up with hydroentanglement process are the most common material used.

Other than hydroentanglement process, there are many materials proposed to further enhance the liquid holding capacity (absorption capacity) of the substrate. For example Chinese patent CN 203496359 U proposes a new structure of substrate for cosmetic applications where sponge particles are sandwiched between two layers of nonwovens so that the substrate can retain a maximum amount of formulation, but this patent doesn't describe possibility of transferring the liquid from substrate to skin.

Although synthetic fibers such as polyester, Nylon, Polypropylene, polyethylene have good capacity for releasing the liquid absorbed by them but at the same time the absorbency of such fibers is very poor.

The use of only Bicomponents fibers for cosmetic application is not so common but blend with cellulosic fibers using hydroentanglement process has been tried. Cellulosic fiber provides good liquid absorption capacity while bi-component fibers contribute to more stechability and surface area.

U.S. Pat. No. 8,911,753 B2 proposes a nonwoven sheet for cosmetic products which is made by uniform blend of hydrophilic fibers and splittable conjugate fibers. The proposed material is claimed to have liquid absorption capacity of 550 to 2000% and having good fitting properties on face.

Japanese Patent No. 3944526 proposes two layer structures for cosmetic products, in which one layer is made up of hydrophilic materials while another layer close to skin is made up of splittable conjugate fiber in order to provide better adhesion to skin surface.

In these described patents the advantage of using bicomponent fibers is better surface area but still hydrophilic fibers are required to hold the liquid and these hydrophilic fibers have limited capacity of releasing the liquid to the skin.

Secondly, the use of these bicomponet fibers in combination with hydrophilic materials using hydroentanglement process is not cost effective for cosmetic applications.

U.S. Pat. No. 5,622,776 relates to hydrophilic glass fibers formed by coating glass fibers with a blend of an amine-aldehyde with an acrylic. Such fibers are used in evaporative cooler pads produced by coating glass fibers with a blend of an acrylic and an amine-aldehyde.

U.S. Pat. No. 7,290,668 relates to a bicomponent fiber wick for use in processing an analyte fluid. The bicomponent fiber wick comprises a self-sustaining, fluid transmissive body comprising a plurality of bicomponent fibers having a fiber structure comprising a first fiber component formed from a polyamide material and a second fiber component.

However U.S. Pat. No. 5,622,776 and U.S. Pat. No. 7,290,668 do not provide components for the cosmetic industry and more generally for products to be in contact with a keratin material, and especially the skin. In particular, glass fibers cannot be used in cosmetic applications.

### SUMMARY OF THE INVENTION

Accordingly, one aim of the present invention is to provide a material, in particular a cosmetic or dermatological material, for cosmetic or dermatological applications.

One aim of the present invention is to provide a material, in particular a cosmetic or dermatological material, having a good absorption capacity of at least one ingredient or composition. A good absorption capacity of at least one ingredient or composition by said material is important notably for storing a sufficient quantity of said at least one ingredient or composition to be released during use of said material.

One aim of the present invention is to provide a material, in particular a cosmetic or dermatological material, having a good delivery capacity, i.e. releasing a sufficient amount of ingredient or composition to a keratin material during the use thereof.

One aim of the present invention is to provide a material, in particular a cosmetic or dermatological material which may release a sufficient amount of ingredient or composition to a keratin material during the use thereof upon an external stimulus, and for example under pressure applied on the material.

One aim of the present invention is to provide a material, in particular a cosmetic or dermatological material which can be stored in sachet with lesser dripping of at least one ingredient or composition absorbed by the material in compare to conventional material.

Also one specific aim of the present invention is to provide a material, in particular a cosmetic or dermatological material, having a good delivery capacity of a skin benefit ingredient

One aim of the present invention is to provide a material, in particular a cosmetic or dermatological material which can be folded easily, and in particular which can be unfolded easily. More particularly, one aim of the present invention is to provide a material, in particular a cosmetic or dermatological material which can be unfolded more easily than conventional cellulosic materials.

One aim of the present invention is to provide a material, in particular a cosmetic or dermatological material which can be prepared according to a cost effective manufacturing process. More particularly, one aim of the present invention is to provide a material, in particular a cosmetic or dermatological material having a low grammage (expressed in grams per square meter (g/m²), thereby representing a low density of the material.

One aim of the present invention is to provide a material, in particular a cosmetic or dermatological material which limits wastage of the ingredient or composition to be delivered to a keratin material, especially to skin.

It is not easy to solve one or more of the technical problems recited above.

### DETAILED DESCRIPTION OF THE INVENTION

The present invention provides a technical solution to one or more technical problems recited above.

The invention relates to a nonwoven solid material preferably a cosmetic or dermatological nonwoven solid material, comprising a plurality of fibers, wherein said fibers comprise a body comprising at least one polymeric material and a body coating comprising at least one hydrophilic polymer, said nonwoven solid material further comprising at least one releasable ingredient or composition, and in particular at least one releasable cosmetic ingredient or composition.

Advantageously, said fibers are entangled.

In one embodiment, said fibers are conjugate fibers comprising a body made from at least two polymeric materials and a body coating comprising at least one hydrophilic polymer.

In one embodiment, said body comprises at least one polymeric material forming the core of the body, at least one polymeric material forming a sheath of the body, and at least one hydrophilic polymer forming said body coating.

In one embodiment, said fibers are bi-component fibers (body of the fibers) with a hydrophilic polymer coating (body coating).

In one embodiment, said body comprises two polymers, preferably with both polymers within the cross section of same fiber. In one embodiment, the polymers of the body in cross section of the fiber are arranged as sheath/core side by side. In one embodiment, the polymers of the body in cross section of the fiber are arranged as concentric or eccentric sheath/core.

In one preferred embodiment, said fiber comprises a body having a higher melting point (melting temperature) than the sheath of the body. Within the meaning of the invention, the melting point corresponds to the temperature of the most endothermic peak observed by thermal analysis (DSC) as described in ISO 1 1357-3; The melting point can be measured using a differential scanning calorimeter (DSC), for example the calorimeter sold under the name “MDSC 2920” by TA Instruments.

In one embodiment, the melting point of the core of the body is higher than 150° C. In one embodiment, the melting point of the core of the body is higher than 230° C.

In one embodiment, the melting point of the core of the body ranges from 150° C. to 300° C.

In one embodiment, the melting point of the core of the body ranges from 230° C. to 300° C.

In one embodiment, the melting point of the sheath of the body is lower than 230° C. In one embodiment, the melting point of the sheath of the body is lower than 150° C. In one embodiment, the melting point of the sheath of the body ranges from 100° C. to 230° C. In one embodiment, the melting point of the sheath of the body ranges from 100° C. to 150° C.

In one preferred and specific embodiment, said fiber comprises a body consisting of a first polymer forming the core of the body, said body consisting of a second polymer forming the sheath of the body, wherein said first polymer has a melting point higher than the second polymer.

Non-limiting examples of polymers which can be used as one of the polymer of said body are selected from the group consisting of Polyester, derivatives of polyester, Nylon6, Nylon 66, Polypropylene, Poly ethylene, Polylactic acid, Polystyrene, and any mixtures thereof.

In one embodiment, non-limiting examples of polymers which can be used as one of the polymer of said body are selected from the group consisting of low density polyethylene (LDPE), polypropylene(PP) high density polyethylene (HDPE), ultra-high molecular weight polyethylene (UHMW), polypropylene (PP), polyvinylidene fluoride (PVDF), polytetrafluoroethylene (PTFE), polyester, and polyethersulfone (PES), and any mixtures thereof.

In one embodiment, the core of the body is selected among polypropylene (PP) and the sheath of the body is selected among polyethylene (PE), wherein said polypropylene has a melting point higher than said polyethylene.

Some common examples of combination of two fibers are shown below:
Polyester Core (250° C. melting point) with Copolyester Sheath (melting points of 110° C. to 220° C.)
Polyester Core (250° C. melting point) with Polyethylene Sheath (130° C. melting point)
Polypropylene Core (175° C. melting point) with Polyethylene Sheath (130° C. melting point

Preferred hydrophilic polymers are those that provide a high absorption capacity, and preferably absorb more than 30 times, and even more preferably more than 50 times, its dry weight when dipped into an aqueous liquid formulation.

The hydrophilic polymer is preferably selected from the group consisting of any commercially available hydrophilic polymers. Non-limiting examples for such polymers made up of acrylic acid, methaacrylic acid, glutamic acid, β-carboxyethyl acrylate, maleic acid, monoesters of maleic acid etc. in one embodiment, said hydrophilic polymers are acrylate polymers.

Acrylate polymers containing carboxyl groups are commercially available. In one particular embodiment, said hydrophilic polymer is poly(acrylic acid). For example, Poly(acrylic acid) can be obtained from, among other sources, Polycryl AG, Bohler, Postfach, CH-6221 Rickenbach, Switzerland (trade name: Polycryl); Stockhausen, 2401 Doyle Street, Greensboro, N.C., 27406-2911; and BFGoodrich, Four Coliseum Centre, 2730 West Tyvola Rd., Charlotte, N.C. 28217-4578 (trade name: Carbopol).

The polymer of the invention (either for the body or the body coating) designates a polymer made on the basis of a single monomer or of two or more monomers. Accordingly, the term polymer is broad in the sense of the invention and includes homopolymers and copolymers except indicated otherwise. Copolymers designate random and alternating copolymers, without limitation except that it is suitable for the invention.

The polymer of the invention may be linear or branched.

In one specific embodiment, the fibers of the invention comprise a PP core and a PE sheath in the body and poly(acrylic acid) in the body coating.

In one specific embodiment, the fibers of the invention consist of a body consisting of a PP core and a PE sheath and of a body coating consisting of poly(acrylic acid).

In a particular embodiment, the solid substrate is a face mask.

In one embodiment, said nonwoven solid material comprises at least one a releasable ingredient or composition providing a skin benefit to a keratin material.

The invention also relates to a face mask comprising a nonwoven solid material as defined in the present invention. In one embodiment, the solid substrate is a wipe.

In one embodiment, the solid substrate has a rounded shape, for example a circular or oval shape. In one embodiment, the solid substrate has a polygonal shape.

In one embodiment, the solid substrate is a face mask capable of defining at least one central through opening, intended to receive a nose of the user, and at least two upper through openings for placing in front of the eyes of the user.

Advantageously, said solid substrate comprises at least two through openings intended to be placed facing the eyes of a user, and another through opening intended to be placed facing the mouth of a user. In a particular embodiment, the face mask may also comprise a slit delimiting a flapper intended to be pushed away by the nose of the user, to delimit a nose insertion through opening. In a variant, the slit delimits a through opening without flapper.

In a particular embodiment, the solid substrate is deformable to the touch, to adapt to the conformation of the body surface.

The size and shape of the solid substrate depends on the application area. For example, a solid substrate adapted to fit the face of a user may have a surface area ranging from 0.25 cm² to 500 cm², preferably from 200 cm² to 450 cm². The solid substrate typically has a density (grammage) of 5 g/m² to 400 g/m², preferably 5 g/m² to 80 g/m².

In one specific embodiment, the nonwoven solid material has a density (grammage) of 5 to 40 g/m², more preferably 10 to 30 g/m², and even more preferably 15 to 25 g/m². Gram/square meter or g/m² is also known as gsm.

One technical advantage of the present invention is the light weight of the nonwoven material.

Advantageously, the solid substrate is porous. Typically, the substrate is porous because of its construction. For example, the nonwoven substrate is porous because of pores formed in between the nonwoven fibers. In one embodiment, the nonwoven solid material comprises body fibers made with fibers having a linear mass density ranging from a 0.1 to 100 dtex, preferably from 0.3 to 10 dtex, even more preferably from 0.5 to 8 dtex.

The invention also relates to a process for manufacturing a nonwoven solid material as defined in the present invention, said process comprising (i) extruding at least one polymeric material, (ii) forming a plurality of fibers comprising said at least one polymeric material, (iii) coating said fibers by at least one hydrophilic polymer, and (iv) bonding said plurality of fibers by a bonding process.

In one embodiment, said bonding process (iv) comprises carding and thermal bonding of said plurality of fibers.

In one embodiment, said process comprises (i) extruding at least two polymeric material in the same spinneret and (ii) forming a plurality of fibers comprising said at least two polymeric materials, one polymeric material forming the core of the fiber body and another polymeric material forming the sheath of the fiber body.

In one embodiment, the process comprises carding said a plurality of fibers comprising said at least two polymeric materials passing hot air through the nonwoven web of fibers. It is referred to hot air process or thermal bonding process.

In a preferred embodiment, the temperature for hot air process for making a nonwoven solid material is selected in a way that only the sheath of the fiber body is melt and the core of the fiber body remains in non-melted form. Accordingly, said thermal bonding is advantageously performed at a temperature for melting the sheath of the fiber body but not melting the core of the body fiber.

In one embodiment, said bonding process (iv) is performed at a temperature in the range of 100° C. to 230° C.

Advantageously, this hot air process results in a consoled material, and gives the nonwoven solid material fluffy a soft feel required in particular for topical applications, especially for cosmetic applications. Because of non-melting of fiber body according to the invention, fiber remains in open structure, which gives it advantageously a fluffy and soft feel.

The invention relates to a nonwoven solid material obtainable by a process as defined in the present invention.

### Applications

The nonwoven solid material can be used as a solid substrate for cosmetic applications.

In one embodiment, said process comprises folding said nonwoven solid material, impregnating said nonwoven solid material in liquid and packing said nonwoven solid material in a sachet.

The invention also relates to a cosmetic article comprising at least one nonwoven solid material or a face mask as defined in the present invention.

In one embodiment the article comprises a packaging, for example a sachet, comprising one or more nonwoven solid substrate of the invention.

In one specific embodiment, the nonwoven solid material can be used as a solid substrate for hydration of a keratin material, and preferably of skin.

The invention relates to a cosmetic use of a nonwoven solid material comprising entangled fibers, wherein said fibers comprise a body comprising at least a polymeric material and a body coating comprising at least one hydrophilic coating.

The invention relates to a cosmetic process for providing a skin benefit to a keratin material, comprising the application to the surface of said keratin material of at least one cosmetic article or a face mask as defined in the present invention, said nonwoven solid material comprising at least one ingredient or composition providing a skin benefit to a keratin material, said nonwoven solid material releasing said ingredient or composition to said keratin material, wherein said keratin material is preferably skin, in particular skin face.

In the figures:

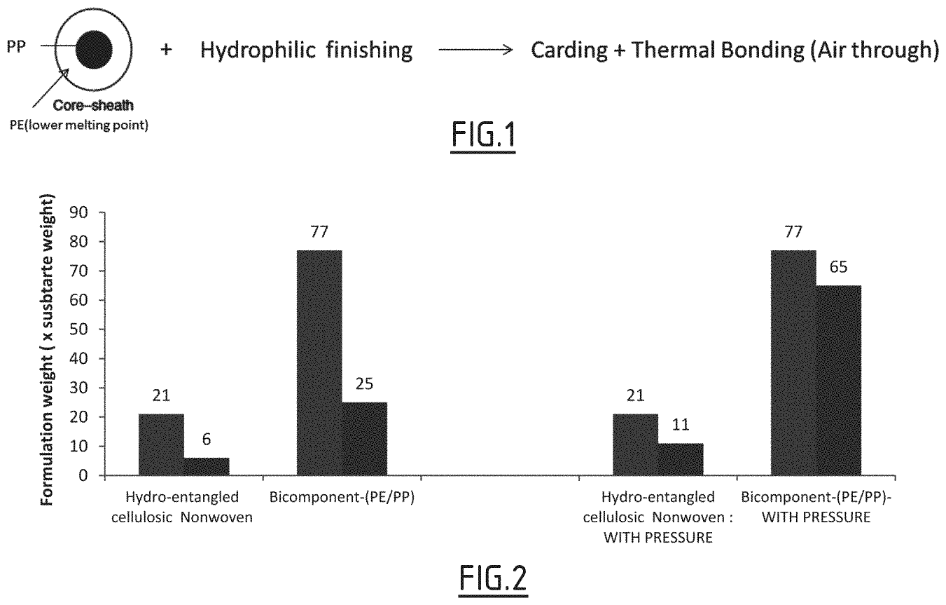

FIG. 1 schematically represents a fiber according to the present invention.

FIG. 2 is a graphic representing the absorption capacity and the delivery capacity of the nonwoven solid material according to the invention versus comparative fibers.

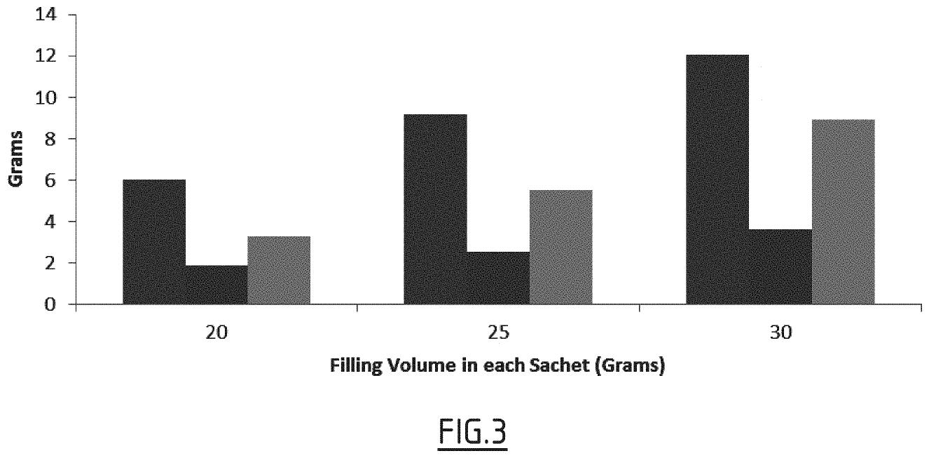

FIG. 3 is a graphic representing the dripping of the nonwoven solid material according to the invention versus comparative nonwoven solid material when the fibers are filled by a liquid composition for topical and cosmetic applications.

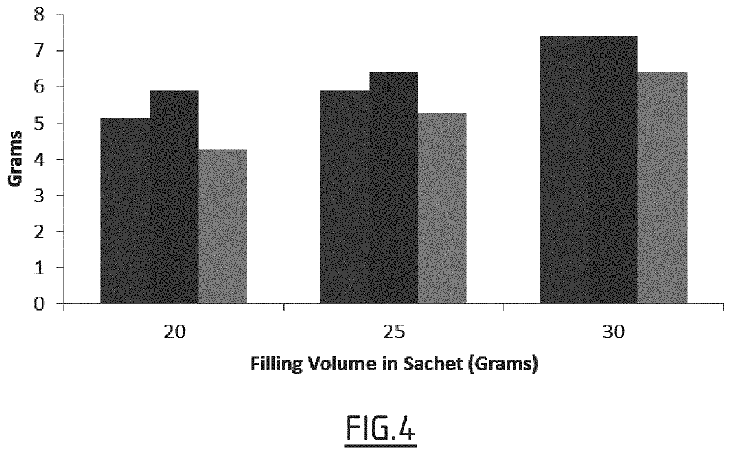

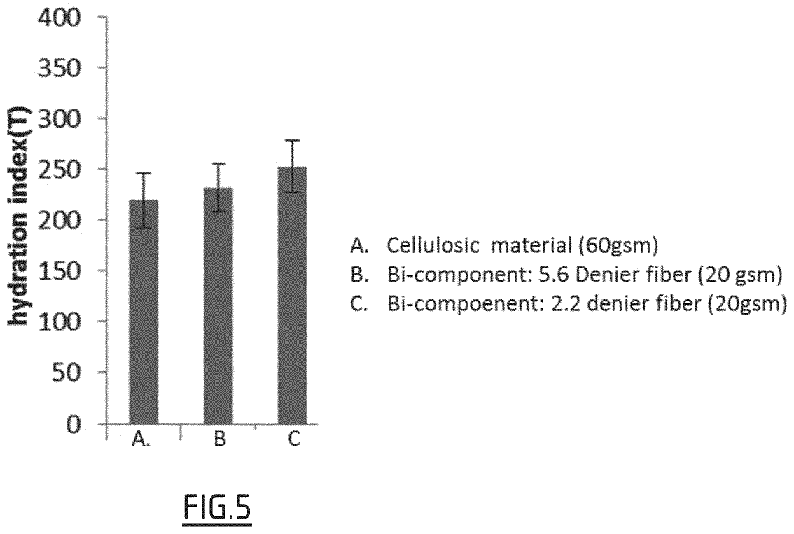

FIG. 4 is a graphic representing the delivery to skin of a liquid composition for topical and cosmetic applications by the nonwoven solid material according to the invention versus comparative nonwoven solid material.

FIG. 5 is a graphic representing the hydration value of a liquid composition for topical and cosmetic applications by the nonwoven solid material according to the invention versus comparative nonwoven solid material.

### EXAMPLES OF INVENTION

### Example 1

### Preparation of a Nonwoven Solid Material According to the Invention with Bi-Component Fibers Having a Core & Sheath Structure

This example of invention relates to use of a bi-component fiber having a core & sheath eccentric structure, thereby forming the body of the fiber used to prepared a nonwoven solid material according to the invention. In this structure, the core is composed of polypropylene and the sheath is composed of polyethylene. This bi-component fiber body was treated with an acrylic polymer by coating the body with the acrylic hydrophilic polymer and then converted into a nonwoven solid material having a density of 20 g/m² using a thermal bonded process. The binding temperature used was 125° C., so that sheath part of the fibers was melted but core part was still remaining non-melted and results in fibrous substrate. (FIG. 1)

### Example 2

### Efficacy of a Nonwoven Solid Material—Absorption and Delivery Capacities

In order to test the efficacy of this nonwoven solid material (prepared according to example 1) in terms of absorption and delivery capacities, one cosmetic formulation as shown in Table: 1 was developed: This formulation represents an example of a formulation

TABLE 1

| Name of Ingredient | % (w/w) |
|---|---|
| Water | 89.2 |
| Glycerin | 5 |
| Butylene glycol | 3 |
| Pentylene glycol | 2 |
| Phenoxy ethanol | 0.5 |
| Xanthan Gum | 0.3 |

The absorption and delivery capacities of developed nonwoven solid material of 20 gsm were compared with commercially available 50 gsm Viscose nonwoven made up with hydroentanglement process. The substrates referred to below and on which the tests were performed are on one hand the nonwoven solid materials according to the present invention and on the other hand the comparative materials (Viscose nonwoven made up with hydroentanglement process).

Test Method:
Cut 10 pieces of substrates in 5×5 cm area and dip them in 50 grams of liquid formulation mentioned in table: 1 for 20 minutes.
Take out the substrate from the liquid formulation, wait for 5 seconds to drip out excess liquid formulation and then take weight of wet substrate pieces one by one.
Apply the wet substrate piece on forearm for 15 minutes and then remove substrate from forearm and weigh it.
A=Avg. dry weight of 5×5 cm substrate. (Avg.: average for the 10 pieces)
B=Avg. wet weight of 5×5 cm wet substrate.
C=Avg. weigh of substrate after removing from skin.
Absorption Capacity (grams)=B−C
Delivery Capacity (grams)=B−(A+C)

FIG. 2 shows that nonwoven solid materials according to the invention are able to absorb 77 times of its dry weight (before impregnation by the liquid formulation) compared to 21 times for cellulosic substrate (comparative material). The absorption capacity is represented by left bars on FIG. 2.

Also the delivery capacity of the liquid formulation to skin is higher for the nonwoven solid materials according to the invention than for the comparative material (cellulosic nonwoven) (25 times its dry weight in comparison with 6 times for cellulosic nonwoven). The delivery capacity is represented by right bars on FIG. 2.

This high absorption and high delivery capacity of the material according to the invention make it particularly suitable for topical applications, especially in cosmetic applications, and more particularly as mask applications.

### Example 3

### Efficacy of a Nonwoven Solid Material Under Pressure

The same experiment as example 2 was conducted but under influence of pressure on skin. For this, when the nonwoven solid materials were applied on skin, they were covered with a plastic band around forearm to apply pressure on them. It was observed that under pressure the delivery capacity of both nonwovens enhances but enhancement is more for nonwoven solid materials according to the invention than comparative cellulosic nonwoven materials. The high absorption and even high delivery capacity under pressure make the nonwoven solid materials according to the invention suitable topical applications, especially in cosmetic applications, and more particularly for wipes applications.

### Example 4

### Efficacy of a Nonwoven Solid Material as Mask—Dripping in Packaging and Delivery to Skin

Mostly sheet mask prepared according to example 1 are individually packet in sachet and impregnated with the most common range of impregnation for sheet mask: 15 grams to 35 gram of liquid formulation according to table 1. Some part of liquid formulation dripped in the sachet, and tests were conducted to check the dripping quantity of the liquid formulation in sachet by the nonwoven solid materials according to the invention and the comparative material.

A mask made either by the nonwoven solid materials according to the invention (20 gsm), a 3 layer cellulosic nonwoven material (60 gsm) or a cotton material (43 gsm) was impregnated with 3 different quantities: 20, 25 and 30 grams. These impregnated quantities are referred as filling volume in FIG. 3 (absciss). In FIGS. 3 and 4, the left bars represent the cotton nonwoven material; the right bars the 3 layer cellulosic nonwoven material and the central bars the nonwoven solid materials according to the invention.

These impregnated masks were sealed individually in an aluminum sachet for 48 hours and then were taken out to weigh. FIG. 3 shows that dripping of the liquid formulation is lesser for the nonwoven solid materials according to the invention than for the cotton and 3 layer cellulosic sheets (ordinate).

After taking out the mask from sachet, the mask was applied on face for 15 minutes, and after 15 minutes the formulation transferred to skin was evaluated.

FIG. 4 shows that the nonwoven solid materials according to the invention have a better delivery to skin at least for the materials impregnated with 20 and 25 grams of liquid formulation.

### Example 5

### Efficacy of a Nonwoven Solid Material for Cosmetic Applications

In order to understand the cosmetic benefit of having higher delivery of a cosmetic formulation, the hydration benefit of a mask according to example 4 was tested with a corneometer (model: CM825, which work on the principal skin capacitance). Three kind of materials were test in combination with the formulation of Table: 1:
A. Cellulosic sheet of 60 gsm (comparative nonwoven material),
B. nonwoven solid materials according to the invention made up with 5.6 denier fibers (nonwoven solid materials according to the invention),
C. nonwoven solid materials according to the invention made up with 2.2 denier fibers (nonwoven solid materials according to the invention).

The linear mass density refers to the linear mass density of the fibers of example 1 prior to coating with the acrylic polymer. After coating, the density is not very different.

These two sheets (A and C) were cut in 5×5 cm sample size and impregnated with 2.38 gram of formulation. These impregnated sheets were applied individually on forearm of 10 consumers for 15 minutes. After 15 minutes, hydration of the treated zone was measured using corneometer.

FIG. 5 illustrates that hydration provided by nonwoven solid materials according to the invention is significantly higher than for comparative material (cellulosic nonwoven material).

### Example 6

### Unfolding Capacity of a Nonwoven Solid Material

In addition, a study with three consumers was conducted to understand the difficulty in unfolding wet facial mask prepared as for example 4:
Cotton nonwoven material (43 gsm) (comparative nonwoven material),
Cellulosic three layers material (60 gsm) (comparative nonwoven material),
Nonwoven solid material according to the invention (20 gsm).

The nonwoven materials were double folded and consumers were ask to rank three masks for easiness of unfolding.

The results are illustrated in table 2.

TABLE 2

| Sample | Average (Consumer: 1, 2, 3) |
|---|---|
| Cotton: 43 gsm | 3 (3, 3, 3) |
| Nonwoven solid materials according to | 1 (1, 1, 2) |
| the invention: 20 gsm | |
| Cellulosic 3 layers: 60 gsm | 2 (2, 2, 1) |

Where: 1 = easiest to unfold and 3 = Difficult to unfold

As shown in Table 2, the mask made up with the nonwoven solid materials according to the invention was found to be the easiest to unfold compared to other two nonwoven materials.

1. A nonwoven solid material, preferably a cosmetic or dermatological nonwoven solid material, comprising a plurality of fibers, wherein said fibers comprise a body comprising at least one polymeric material and a body coating comprising at least one hydrophilic polymer, said nonwoven solid material further comprising at least one releasable cosmetic ingredient or composition.

2. The nonwoven solid material according to claim 1, wherein said fibers are conjugate fibers comprising a body made from at least two polymeric materials and a body coating comprising at least one hydrophilic polymer.

3. The nonwoven solid material according to claim 1, wherein said body comprises at least one polymeric material forming the core of the body, at least one polymeric material forming a sheath of the body, and at least one hydrophilic polymer forming said body coating.

4. The nonwoven solid material according to claim 1, wherein said nonwoven solid material comprises at least one a releasable ingredient or composition providing a skin benefit to a keratin material.

5. A face mask comprising said nonwoven solid material according to claim 1.

6. A process for manufacturing a nonwoven solid material according claim 1, said process comprising (i) extruding at least one polymeric material, (ii) forming a plurality of fibers comprising said at least one polymeric material, (iii) coating said fibers by at least one hydrophilic polymer, and (iv) bonding said plurality of fibers by a bonding process.

7. The process according to claim 6, wherein said bonding process (iv) comprises carding and thermal bonding of said plurality of fibers.

8. The process according to claim 7, wherein said thermal bonding is performed at a temperature for melting the sheath of the fiber body but not melting the core of the body fiber.

9. The process according to claim 5, wherein said process comprises folding said nonwoven solid material, impregnating said nonwoven solid material in liquid and packing said nonwoven solid material in a sachet.

10. A cosmetic article comprising at least one nonwoven solid material according to claim 1.

11. A method for applying a cosmetic providing a skin benefit comprising applying the cosmetic with a nonwoven solid material comprising entangled fibers, wherein said fibers comprise a body comprising at least a polymeric material and a body coating comprising at least one hydrophilic coating.

12. A cosmetic process for providing a skin benefit to a keratin material, comprising the application to the surface of said keratin material of at least one cosmetic article according to claim 10, said nonwoven solid material comprising at least one ingredient or composition providing a skin benefit to a keratin material, said nonwoven solid material releasing said ingredient or composition to said keratin material, wherein said keratin material is preferably skin, in particular skin face.

13. A cosmetic article comprising a face mask according to claim 5.

14. A cosmetic process for providing a skin benefit to a keratin material, comprising the application to the surface of said keratin material of a face mask according to claim 5, said nonwoven solid material comprising at least one ingredient or composition providing a skin benefit to a keratin material, said nonwoven solid material releasing said ingredient or composition to said keratin material, wherein said keratin material is preferably skin, in particular skin face.
